(19) United States
(12) Patent Application Publication
(10) Pub. No.: US 2025/0367015 A1
(43) Pub. Date: Dec. 4, 2025

(54) SUPPORT VEST FOR PEDIATRIC PATIENTS

(71) Applicant: Imam Abdulrahman Bin Faisal University, Dammam (SA)

(72) Inventors: Hessa ALLAYLI, Dammam (SA); Ruba ALKHALDI, Dammam (SA); Hala EL-WAKEEL, Dammam (SA); Zati HAZIRAT, Dammam (SA)

(73) Assignee: Imam Abdulrahman Bin Faisal University, Dammam (SA)

(21) Appl. No.: 18/972,136

(22) Filed: Dec. 6, 2024

Related U.S. Application Data
(60) Provisional application No. 63/654,181, filed on May 31, 2024.

(51) Int. Cl. A61F 5/02; B63C 9/115

(52) U.S. Cl. CPC A61F 5/026; B63C 9/115

(57) ABSTRACT

A pediatric positioning apparatus that includes a support vest and a neck support. The support vest includes a network of rigid support members and a cover, a pair of waist supports configured to wrap around the waist a patient, and a pair of anterior straps attached configured to pass over shoulders and chest of a patient and secure to the waist supports. The neck support includes a posterior neck strap and an anterior support jaw including a superior jaw arc that includes a chin contact portion and an inferior jaw arc comprising a chest contact portion. The anterior support jaw is rigid and configured to contact a chin and a chest of a patient such that the anterior support jaw supports the chin of the patient in a ventral direction and prevents contact between the chin and the chest of the patient.

### CROSS-REFERENCE TO RELATED APPLICATIONS

The present application claims priority to U.S. Provisional Application No. 63/654,181 filed May 31, 2024, which is incorporated by reference in its entirety for all purposes.

### BACKGROUND OF THE INVENTION

### Field of the Invention

The present disclosure relates to a pediatric positioning apparatus that takes the form of a supportive vest with a neck support.

### Discussion of the Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Many infants, toddlers, and children suffer from conditions which cause them to require significant additional care. Motor and neurological conditions can make it difficult or impossible for children to sit or stand without support. Muscle weakness, lack of coordination, paralysis, and other factors can prevent a child from performing normal activities unaided. The relatively large head size compared to body size of children can make even reclining difficult without additional external support. This can lead to great difficulty for the child and their caretaker when performing daily tasks such as walking, sitting, traveling such as being carried, bathing, and other activities.

For example, Sandhoff disease is a rare inherited disorder that progressively destroys nerve cells (neurons) in the brain and spinal cord (central nervous system). This condition is classified into three major types based on the age at which signs and symptoms first appear: infantile, juvenile, and adult. The infantile form of Sandhoff disease is the most common and severe form and becomes apparent in infancy. Infants with this disorder typically appear normal until the age of 3 to 6 months, when their development slows and muscles used for movement weaken. Affected infants lose motor skills such as turning over, sitting, and crawling. As the disease progresses, children with Sandhoff disease experience seizures, vision and hearing loss, and intellectual disability.

Because of the severity and nature of the condition, Sandhoff disease patients require round-the-clock care. However, because of the loss of motor function, Sandhoff disease patients can be unable to support themselves or parts of their bodies even while receiving direct assistance or care from a caregiver. This causes a dramatic increase in the risk of injury to a patient during routine activities such as sitting, being carried, bathing, and the like. This also places an increased burden on caregivers. A caregiver must provide a dramatically increased level of support to a child with Sandhoff disease to ensure the child receives the care and assistance they need.

A typical method of assisting such children and their caregivers is to use a positioning or support device. However, many such devices are large, bulky, or immobile devices that are designed to have a patient or child placed in them. As such, it is very difficult to move the patient and device together, the patient must be removed from the device and transferred unsupported to another device, or a separate device is required for different positions or activities.

The present disclosure provides a wearable supporting prosthetic that is wearable by a pediatric patient. The wearable support prosthetic is capable of being used in a variety of activities and situations, including during bathing.

### SUMMARY OF THE INVENTION

The present disclosure relates to a pediatric positioning apparatus, comprising a support vest that includes a vest posterior portion comprising a network of rigid support members and a vest posterior portion cover, a pair of waist supports attached to the vest posterior portion and configured to wrap around at least 50% of a waist circumference of a patient, and a pair of anterior straps attached to the vest posterior portion and configured to pass over shoulders and chest of a patient and secure to the waist supports. The pediatric positioning apparatus also comprises a neck support that includes a posterior neck strap and an anterior support jaw comprising a superior jaw arc comprising a chin contact portion and an inferior jaw arc comprising a chest contact portion, the anterior support jaw being rigid and configured to contact a chin and a chest of a patient such that the anterior support jaw supports the chin of the patient in a ventral direction and prevents contact between the chin and the chest of the patient.

In some embodiments, the rigid support members are formed of a plastic.

In some embodiments, the rigid support members have a flattened contact surface, the contact surface being the surface oriented toward a patient wearing the support vest.

In some embodiments, the rigid support member has a support ridge disposed on a surface opposite the flattened contact surface.

In some embodiments, the network of rigid support members comprises a peripheral support member disposed about a periphery of the network of rigid support members.

In some embodiments, the vest posterior portion cover is formed of a textile.

In some embodiments, the textile is a synthetic textile formed of a polymer.

In some embodiments, the network of rigid support members is configured to be removable from the vest posterior portion cover.

In some embodiments, each waist support includes a rigid waist support member.

In some embodiments, the pediatric positioning apparatus, further comprises a waist belt attached to the pair of waist supports and configured such that the waist belt and pair of waist supports together wrap around an entirety of a waist circumference of a patient.

In some embodiments, the waist belt is attached to the pair of waist supports using hook-and-loop fasteners.

In some embodiments, the neck support is connected to the support vest by at least one selected from the group consisting of the anterior straps, a neck support connection strap attached to the support vest and the neck support, and a rigid neck support connection member attached to the support vest and the neck support.

In some embodiments, the anterior support jaw is formed of a plastic.

In some embodiments, the posterior neck strap is formed of a textile.

In some embodiments, the textile is a synthetic textile formed of a polymer.

In some embodiments, the pediatric positioning apparatus further comprises a posterior head support attached to a superior end of the vest posterior portion, the posterior head support configured to provide support to a head of a patient in a dorsal direction.

In some embodiments, the posterior head support comprises a bathing support member configured to permit a patient to be placed in a reclining position in a bathing apparatus while contacting the bathing apparatus to prevent the patient from being submerged in the bathing apparatus.

In some embodiments, the posterior head support comprises a rigid posterior head peripheral support member disposed about a periphery of a flexible posterior head support platform.

In some embodiments, the rigid posterior head peripheral support member is formed of a plastic and the flexible posterior head support platform is formed of a textile.

In some embodiments, the pediatric positioning apparatus is devoid of electronic sensors.

### BRIEF DESCRIPTION OF THE DRAWINGS

FIG. 1A shows a rear three-quarters view of a support vest according to an exemplary embodiment of the present disclosure.

FIG. 1B shows a rear three-quarters view of a support vest with a portion of the vest posterior portion cover removed showing the network of rigid support members according to an exemplary embodiment of the present disclosure.

FIG. 1C shows a schematic depiction of a frontal view of a support vest according to an exemplary embodiment of the present disclosure.

FIG. 1D shows a schematic depiction of a side view of a support vest according to an exemplary embodiment of the present disclosure.

FIG. 2A shows a front three-quarters view of a neck support according to an exemplary embodiment of the present disclosure.

FIG. 2B shows a rear three-quarters view of a neck support according to an exemplary embodiment of the present disclosure.

FIG. 2C shows a schematic depiction of a top-down view of a neck support according to an exemplary embodiment of the present disclosure.

FIG. 2D shows a schematic depiction of a side view of a neck support according to an exemplary embodiment of the present disclosure.

FIG. 2E shows a schematic depiction of a frontal view of a neck support according to an exemplary embodiment of the present disclosure.

FIG. 3A shows a three-quarters view of a posterior head support according to an exemplary embodiment of the present disclosure.

FIG. 3B shows a schematic depiction of a top-down view of a posterior head support according to an exemplary embodiment of the present disclosure.

FIG. 3C shows a schematic depiction of a side view of a posterior head support according to an exemplary embodiment of the present disclosure.

FIG. 4 shows a pediatric patient wearing a pediatric position apparatus according to an exemplary embodiment of the present disclosure.

### DETAILED DESCRIPTION OF THE INVENTION

In the following description, it is understood that other embodiments may be utilized and structural and operational changes may be made without departure from the scope of the present embodiments disclosed herein.

### Definitions

As used herein the words “a” and “an” and the like carry the meaning of “one or more.”

As used herein, the terms “optional” or “optionally” means that the subsequently described event(s) can or cannot occur or the subsequently described component(s) may or may not be present (e.g., 0 wt. %).

As used herein, the term “textile” refers to a fiber-based material, including fibers, yarns, filaments, threads, fabrics, and the like. A textile can be defined or identified by a combination of factors including fiber, yarn, construction, processing, and finishing. Typically, fiber refers to the smallest unit of material while yarn refers to a collection of fibers which can be spun, twisted, felted, or grouped into a larger unit for inclusion in a textile. A textile can be woven or non-woven.

According to a first aspect, the present disclosure relates to a pediatric positioning apparatus. The pediatric positioning apparatus comprises a support vest. The support vest includes a vest posterior portion comprising a network of rigid support members (“network”).

In some embodiments, the rigid support members are formed of a plastic. In general, any suitable type of plastic known to one of ordinary skill in the art may be used. Examples of plastics include, but are not limited to acrylonitrile butadiene styrene (ABS), polystyrene (including high impact polystyrene), polycarbonate, polyolefins such as polyethylene, polypropylene, polymethylpentene, polybutene, ethylene-octene copolymers, propylene-butane copolymers, and mixtures of these, polymethyl methacrylate (PMMA) (also called acrylic), aramids, polyetheretherketone (PEEK), polyetherimide (PEI), polytetrafluoroethylene (PTFE), polyvinylchloride (PVC), Polyamide-imide (PAI), and mixtures of these. A plastic may contain particulate, fibrous or wire reinforcements. Preferably, the plastic has some flexibility and resiliency so as to make it durable and not easily cracked or broken. In some embodiments, the plastic is a medical grade plastic. A medical grade plastic can be any plastic safe for use in a medical device. For example, a medical grade plastic can be a plastic having a United States Pharmacopoeia plastic designation of Class I, II, III, IV, or V or any plastic which conforms to ISO 10993 standards for biocompatibility. For example, a medical grade plastic can have reduced levels or be devoid of certain components such as plasticizers, pigments, and processing aids, or can include certain additives such as antimicrobial agents or antioxidants.

In some embodiments, a rigid support member has a flattened contact surface. The contact surface refers to a surface which forms an interior or patient-facing surface of the network of rigid support members. In some embodiments, the network is formed of rigid support members having the same shape. In some embodiments, the network is formed of rigid support members having different shapes. Preferably, any patient-facing surface of any rigid support member is flattened. This flattening may be advantageous for avoiding sharp points, ridges, corners, or other projections that may injure or cause discomfort to a patient wearing the apparatus (“wearer”). In some embodiments, the non-patient-facing surfaces of each rigid support member is devoid of sharp points, ridges, corners, or other projections.

In some embodiments, a rigid support member can have a substantially flattened elongated shape. For example, a rigid support member can have a flattened elongated ellipse shape or a flattened elongated rectangle shape having rounded corners. Such a flattened elongated shape can have a length that is at least 5 times, preferably at least 7.5 times, preferably at least 10 times a width of the flattened elongated shape. Such a flattened elongated shape can have a thickness which is less than ⅕^{th}, preferably less than 1/10^{th}, preferably less than 1/15^{th}, preferably less than 1/20^{th}, preferably less than 1/50^{th} of a length of the flattened elongated shape.

In some embodiments, the rigid support member has a stiffening ridge disposed on a surface opposite the flattened contact surface. The contact surface can be the surface oriented toward a patient wearing the support vest. In general, the stiffening ridge is a structure which increases a flexural and/or torsional stiffness of the rigid support member. In some embodiments, the stiffening ridge can be oriented substantially perpendicular to a plane of the flattened contact surface. In some embodiments, the stiffening ridge can be a thickened portion of the rigid support member. In some embodiments, a rigid support member having a stiffening ridge has a substantially T-shaped cross-section. In some embodiments, the stiffening ridge can be formed of the same material as the rigid support member. In some embodiments, the stiffening ridge can be formed of a different material from the rigid support member. For example, the stiffening ridge can be formed of a rod of some material, such as a metal, that is enclosed by the material forming the rigid support member. In some embodiments, the stiffening ridge can include grooves, notches, depressions, indentations, or other similar structures configured to act as intersection points of two or more rigid support members. For example, the grooves or notches in the stiffening ridge can be of a size and orientation to allow one rigid support member to securely lay on top of another rigid support member to allow them to interlock. The stiffening ridge may also be known as a “rib”, “stiffening rib”, or other similar term.

In some embodiments, the flattened contact surface includes grooves, notches, depressions, indentations, or other similar structures configured to act as intersection points of two or more rigid support members. For example, the grooves or notches in the flattened contact surface can be of a size and orientation to allow one rigid support member to securely lay on top of another rigid support member to allow them to interlock. For example, the grooves or notches in the flattened contact surface can be configured to be aligned with the grooves or notches disposed in a stiffening rib of another rigid support member.

In general, the rigid support members can be arranged in any suitable manner to form the network. For example, the rigid support members can be arranged in a grid. In general, the grid can be defined or identified by a shape formed by the space between rigid support members. For example, the grid can be a rectangular grid, square grid, triangular grid, hexagonal grid, rhombohedral grid, or other such grid. The grid can be a regular grid or an irregular grid. As used herein, the term “regular grid” refers to a grid which the size and shape of each shape formed by the space between rigid support members is the same. The term “irregular grid” refers to a grid in which each shape formed by the space between rigid support members does not have the same size and shape. For example, there may be two or more types of shapes formed by the space between rigid support members, there may be similar shapes formed by the space between rigid support members that have different sizes, or both.

In some embodiments, the network can be formed by overlapping rigid support members. In some embodiments, the overlapping involves a first layer of rigid support members substantially oriented in a first direction (e.g., horizontal support members) and a second layer of rigid support members substantially oriented in a second direction (e.g., vertical support members). In some embodiments, the network includes fasteners disposed at intersections between two or more rigid support members. In general, any suitable type of fastener can be used, such as rivets, snaps, buttons, press-fit fasteners, and the like. In some embodiments, the rigid support members are connected at intersection points by meld welding. In some embodiments, the network is formed by weaving. That is the rigid support members can for the network by having a single rigid support member passing over top of certain other rigid support members and beneath certain other rigid support members to form a woven pattern. In general, the woven pattern can include any suitable type of weave, such as plain weave, twill weave, satin weave, or combinations thereof. In some embodiments, a rigid support member can include projections configured to fit into the grooves or notches of another rigid support member to interlock the rigid support members.

In some embodiments, the network of rigid support members comprises a peripheral support member disposed about a periphery of the network of rigid support members. The peripheral support member can serve to secure the rigid support members such that the network remains intact (i.e., does not come apart). In some embodiments, the peripheral support member is disposed about an entirety of the periphery of the network. In general, the peripheral support member can take any suitable form and be constructed of any suitable material. For example, the peripheral support member can be substantially similar to the rigid support members used to form the network. The rigid peripheral support can be disposed about a periphery of the network such that it is connected to the ends of two or more rigid support members. In some embodiments, the peripheral support member can be formed of a textile. A textile peripheral support member can be sewn around a periphery of the network to prevent the rigid support members from leaving the network. In general, any number of peripheral support members can be used. In some embodiments, one peripheral support member is used. Such a single peripheral support member can encompass an entirety of the periphery of the network. In some embodiments, a plurality of peripheral support members are used.

The vest posterior portion also includes a vest posterior portion cover. The vest posterior portion cover can also be referred to as a “cover”, “vest cover”, or other similar term. In some embodiments, the vest posterior portion cover is formed of a textile. In general, any suitable textile may be used. The textile may be a natural textile. That is, the textile is formed from fibers which are derived from natural sources, such as animals or plants. Such fibers may be treated, processed, or otherwise altered prior to use or as part of forming the textile. The textile may be an artificial textile. That is, the textile is formed from fibers which are synthetic or not derived from natural sources, such as man-made polymer fibers. In some embodiments, the textile is a synthetic textile formed of a polymer. Examples of such suitable textiles or fibers include, but are not limited to cotton, canvas, wool, silk, linen, jute, modal, alpaca, olefin fiber, camel hair, hemp, horsehair, ixtle, flax, bamboo, alginate, rayon, polymers, and mixtures thereof. Examples of applicable polymers may include polyolefin, polyacetal, polyamide, polyester, cellulose ether and ester, polyalkylene sulfide, polyarylene oxide, polysulfone, modified polysulfone polymers, nylon, polystyrene, polyacrylonitrile, polycarbonate, polyvinyl alcohol, polyvinylpyrrolidone, poly(lactic acid), poly(lactic-co-glycolic acid), polybutylene terephthalate, polyurethane, polychloroprene, and mixtures thereof. Examples of textiles made from polymers include, but are not limited to aramid (e.g. Twaron™, Kevlar™, and Nomex™), acrylic, nylon, spandex, ingeo, lurex, neoprene, and carbon fiber.

In general, the textile may be a woven textile or a non-woven textile. A woven textile may be formed from the fibers using any suitable weave, such as plain weave, twill weave, satin weave, or combinations thereof. The woven textile may be formed from threads, yarns, or other material formed from interlocked fibers which are woven to form the woven textile. It should be understood here that for the purposes of the present disclosure “woven textile” includes textile which is formed by knitting. While in a woven textile, there are separate warp and weft threads, knitting has only a single such type of thread which is looped, tied, crossed, bent, entangled, or otherwise interlocked with other threads. For the purposes of the present disclosure, the term “woven textile” refers to knit textiles, woven textiles, and other textiles in which the fibers, threads, or yarns are interlocked or otherwise connected in such an ordered fashion. This is in contrast to a non-woven textile in which the fibers, threads, or yarns are interlocked in a non-ordered fashion. A non-woven textile comprises fibers, threads, or yarns which may be interlocked, entangled, or bonded mechanically, thermally, or chemically. The non-woven textile may comprise an adhesive or a binder. The non-woven textile may be formed using any suitable technique known to one of ordinary skill in the art. For example, the non-woven textile may be a staple non-woven, melt-blown, spunlaid, flashspun, needle-punched, felted, or formed by any other suitable technique.

In some embodiments, the vest posterior portion cover is configured to be used in water. For example, the cover can be constructed in a manner and/or of materials such that the cover is waterproof, water resistant, water repellant, quick-drying, or any combination of these. The support vest may be used in water. For example, the support vest may be used while bathing, swimming, in wet environments, in environments where a wearer may be splashed, or the like, or may be used in environments where the wearer may sweat. Having the cover be waterproof, water resistant, water repellant, and/or quick-drying may be advantageous for wearer comfort, for ease of use, for longevity of the support vest, for cleaning, or for some other purpose. This may be particularly advantageous for use with the bathing support member described below.

In some embodiments, the network of rigid support members is configured to be removable from the vest posterior portion cover. That is, entirety of the network can be removed from the cover. This may be advantageous for cleaning and/or drying of the network, the cover, and/or any other component of the support vest. In general, the network can be secured within the cover using any suitable means known to one of ordinary skill in the art. In some embodiments, the cover can define an interior space in which the network is placed unsecured. The cover can be closed around the interior space, for example by using a fastening means such as a zipper, buttons, snaps, clasps, buckles, and the like. In some embodiments, the network is secured within an interior space defined by the cover. The network can be secured to the cover by having a portion of the cover fasten to or connect to a portion of the network. For example, one half of a zipper can be disposed on the network and the other half can be disposed on the cover such that the network can be secured to the cover by zippering the two halves of the zipper together, the cover can include buttons and the network can include button holes or vice-versa, the cover can include one half of a snap-fit fastener and the network can include the other half, or the like.

The support vest also includes a pair of waist supports attached to the vest posterior portion. In some embodiments, the pair of waist supports is configured to wrap around at least 50%, preferably at least 55%, preferably at least 60%, preferably at least 65%, preferably at least 70%, preferably at least 75% of a waist circumference of a patient (wearer). In some embodiments, the pair of waist supports is disposed symmetrically about a midline of the support vest. That is, each waist support is connected to a posterior portion of the support vest and extends in a forward (ventral) direction. In some embodiments, each waist support extends in a forward direction by an equal amount. That is, each waist support can have the same length or extent around a circumference of the waist. Such an equal length can produce a gap disposed between the waist supports. The gap can be substantially centered on a midline of the support vest. In some embodiments, each waist support extends in a forward direction by an equal amount. That is, each waist support can have a different length or extent around a circumference of the waist. Such an unequal length can produce a gap disposed between the waist supports that is not centered on a midline of the support vest.

In some embodiments, each waist support includes a rigid waist support member. In general, the rigid waist support member can be substantially similar to a rigid support member that forms the network as described above. A rigid waist support member can differ from a rigid support member in terms of any suitable feature or property, such as construction material(s), size, shape, flexural or torsional stiffness, stiffening ridge, flattened contact surface, an the like. In some embodiments, the rigid waist support member can have a flattened contact surface as described above. In some embodiments, the rigid waist support member can have a stiffening ridge as described above. In some embodiments, the rigid waist support member can have a flattened elongated shape as described above.

In some embodiments, each waist support includes a single rigid waist support member. In some embodiments, each waist support includes a plurality of rigid waist support members. In general, any number of rigid waist support members can be used. In some embodiments, each waist support includes a plurality of rigid waist support members arranged into a network. In some embodiments, the network of rigid waist support members is separate from the network of rigid support members included in the vest posterior portion. In some embodiments, the network of rigid waist support members is connected to the network of rigid support members included in the vest posterior portion. Such a connection can be a reversible connection (e.g., one in which the network of rigid waist support members can be separated or removed from the network of rigid support members included in the vest posterior portion), or a permanent connection (e.g., the network of rigid waist support members is integrated with the network of rigid support members included in the vest posterior portion).

In some embodiments, the pediatric positioning apparatus includes a waist support cover disposed on each waist support. In general, a waist support cover can be substantially similar to the vest posterior portion cover as described above. A waist support cover can differ from the vest posterior portion cover in terms of any suitable feature or property, such as construction material(s), size, shape, waterproofing, water resistance, water repelling behavior, drying time, and the like. In some embodiments, each waist support cover is connected to the vest posterior portion cover. Such a connection can be a reversible connection (e.g., one in which a waist support cover can be separated or removed from the vest posterior portion cover), or a permanent connection (e.g., one in which a waist support cover is integrated with the vest posterior portion cover).

In some embodiments, the rigid waist support member is configured to be removable from the waist support. That is, rigid waist support can be removed from the waist support cover. This may be advantageous for cleaning and/or drying of the network, the cover, and/or any other component of the support vest. In general, the rigid waist support member can be removable as described above. In some embodiments, the rigid waist support member is configured to not be removable from the waist support.

In some embodiments, the pediatric positioning apparatus, further comprises a waist belt attached to the pair of waist supports. In some embodiments, the waist belt is configured such that the waist belt and pair of waist supports together wrap around an entirety of a waist circumference of a patient (wearer). That is, the waist belt can extend across a gap created between the waist supports. In some embodiments, the waist belt is attached to the pair of waist supports using a textile fastening implement. Examples of textile fastening implements include, but are not limited to buttons, snaps, hooks, zippers, clasps, buckles, hook-and-loop fasteners, and the like.

The support vest also includes a pair of anterior straps attached to the vest posterior portion and configured to pass over shoulders and chest of a patient (wearer) and secure to the waist supports. In some embodiments, the anterior straps are attached to the vest posterior portion using a textile fastening implement. Examples of textile fastening implements include, but are not limited to buttons, snaps, hooks, zippers, clasps, buckles, hook-and-loop fasteners, and the like. In some embodiments, the pair of anterior straps is attached to the vest posterior portion cover.

In some embodiments, the support vest can include a floatation aid. In general, the floatation aid can be any suitable structure or feature that causes the support vest to float in water. That is, the floatation aid is a buoyant structure or feature. For example, the floatation aid can be a structure formed of or filled with air, foam, or other buoyant material. The floatation aid can be attached to or disposed upon the support vest or the support vest cover. In some embodiments, the floatation aid is reversibly attached to or disposed upon the support vest or support vest cover. That is, the floatation aid can be removed from the support vest or support vest cover. For example, the support vest or support vest cover can include pockets in which one or more floatation aids can be inserted. In general, any number of floatation aids can be used.

The pediatric positioning apparatus comprises a neck support. In some embodiments, the neck support comprises a posterior neck strap. In some embodiments, the posterior neck strap is formed of a textile. In general, the textile may be as described above. In some embodiments, the textile is a synthetic textile. In general, the synthetic textile may be as described above. In some embodiments, the synthetic textile is formed of a polymer, as described above. In some embodiments, the posterior neck strap is formed of an elastic material. That is, the posterior neck strap can be configured to stretch or capable of stretching. In some embodiments, the posterior neck strap can be configured to stretch or capable of stretching in a direction along a length of the posterior neck strap.

In some embodiments, the posterior neck strap can include a textile fastening implement. Examples of textile fastening implements include, but are not limited to buttons, snaps, hooks, zippers, clasps, buckles, hook-and-loop fasteners, and the like. In some embodiments, the posterior neck strap has a slit, buttonhole, opening, fastener, snap, or other suitable means configured to be secured to or about a corresponding feature on another portion of the neck support, such as the anterior support jaw described below. Example of such features include but are not limited to buttons, snaps, posts, hooks, pegs, and the like. In some embodiments, the posterior neck strap includes a plurality of textile fastening implements such that a length or extent of the posterior neck strap can be adjusted. In some embodiments, the posterior neck strap can be unfastened such that one end of the posterior neck strap remains attached to the neck support while the other end of the posterior neck strap is free. This may be particularly advantageous for putting on or taking off the neck support.

In some embodiments, the neck support comprises an anterior support jaw. In some embodiments, the anterior support jaw comprises a superior jaw arc comprising a chin contact portion. In some embodiments, the anterior support jaw comprises an inferior jaw arc comprising a chest contact portion. In some embodiments, the anterior support jaw is rigid. In some embodiments, the anterior support jaw is configured to contact a chin and a chest of a patient (wearer) such that the anterior support jaw supports the chin of the patient (wearer) in a ventral direction and prevents contact between the chin and the chest of the patient (wearer). In some embodiments, the anterior support jaw is formed of a plastic. Preferably, the plastic is a rigid plastic. Examples of suitable plastics include, but are not limited to acrylonitrile butadiene styrene (ABS), polyethylene, polypropylene, polyacrylate, polymethacrylate, poly(methyl methacrylate), polyacrylic acid, poly amide, polybutylene, polycarbonate, polyvinylchloride, and the like.

In some embodiments, the anterior support jaw has a jaw angle of 120° to 10°, preferably 115° to 15°, preferably 110° to 20°, preferably 105° to 25°, preferably 100° to 30°, preferably 95° to 35°, preferably 90° to 40°. The jaw angle is defined as the angle formed between the superior jaw arc and the inferior jaw arc. In some embodiments, the jaw angle is adjustable. In some embodiments, the jaw angle is adjustable to any angle between 120° to 10°, preferably 115° to 15°, preferably 110° to 20°, preferably 105° to 25°, preferably 100° to 30°, preferably 95° to 35°, preferably 90° to 40°.

In some embodiments, the superior jaw arc has a top surface that is planar. In some embodiments, the superior jaw arc has a top surface that is oriented substantially perpendicular to a neck of the patient. That is, a top surface of the superior jaw arc is substantially parallel to a patient's chin. In some embodiments, the superior jaw arc has a shape that includes a curve or valley defined in the center of the superior jaw arc. The curve or valley can be shaped such that the center of the superior jaw arc is lower than the left or right sides of the superior jaw arc. In this way, the patient's chin may rest in the curve or valley of the superior jaw arc. By resting in the curve or valley, the patient's chin may be supported both frontally (i.e., prevented from moving toward the chest) and laterally (i.e., preventing the patient's head from flopping left or right). In some embodiments, a curve or valley shape is defined by raised lateral portions of the superior jaw arc. That is, the center of the superior jaw arc is not lowered, but the side portions are raised to provide lateral stability. The resulting shape can have an angled bottom or “V-shape” or can cave a rounded bottom or “U-shape”. In some embodiments, the curve or valley shape of the superior jaw arc is adjustable. For example, the superior jaw arc can be adjustable from a substantially flat shape to raise the sides of the superior jaw arc to form the V-shape or U-shape. In this way, the superior jaw arc can be adjusted from providing little to no lateral support to a maximum lateral support. This adjustability may be advantageous for adapting the neck support to a variety of circumstances. This adjustability may be achieved through any suitable mechanism known to one of ordinary skill in the art. For example, the superior jaw arc may include a central hinge capable of locking into two or more positions.

In some embodiments, the chest contact portion comprises a chest contact pad. The chest contact pad can be an enlarged portion configured to spread a contact pressure over a larger area of the chest. This spreading may be useful for increased user comfort. In some embodiments, the chest contact pad includes a cushion as described below. In some embodiments, the chest contact pad is substantially flattened. In some embodiments, the chest contact pad is contoured to provide uniform contact to a user's chest.

In some embodiments, the chin contact portion and/or the chest contact portion include a cushion. In general, the cushion can be formed of any suitable material. Examples of suitable materials which may be used for the cushion include, but are not limited to elastomeric materials include but are not limited to rubbers such as natural rubber, nitrile rubber, butyl rubber, styrene-butadiene rubber, EPDM rubber, chloroprene, Neoprene®, hydrogenated nitrile rubber, and the like, silicones such as dimethylsilicone, methyl-phenylsilicone, methylvinylsilicone, mixtures thereof, and the like, fluoroelastomers such as FKM, FFKM, FEPM, and the like. The cushion(s) may be advantageous for increasing wearer comfort while wearing the neck support. In some embodiments, the cushion is disposed on a surface of the chin contact portion and/or the chest contact portion that is in contact with the chin or chest, respectively. That is, a cushion disposed on the chin contact portion is preferably disposed on a surface of the chin contact portion that is actually in contact or configured to contact a wearer's chin.

In some embodiments, the neck support is connected to the support vest by at least one selected from the group consisting of the anterior straps, a neck support connection strap attached to the support vest and the neck support, and a rigid neck support connection member attached to the support vest and the neck support.

In some embodiments, the neck support is configured to be used in water. For example, the neck support or any portion thereof can be constructed in a manner and/or of materials such that the neck support is waterproof, water resistant, water repellant, quick-drying, or any combination of these. For example, the neck support may be used while bathing, swimming, in wet environments, in environments where a wearer may be splashed, or the like, or may be used in environments where the wearer may sweat. Having the neck support be waterproof, water resistant, water repellant, and/or quick-drying may be advantageous for wearer comfort, for ease of use, for longevity of the support vest, for cleaning, or for some other purpose. This may be particularly advantageous for use with the bathing support member described below.

In some embodiments, the pediatric positioning apparatus further comprises a posterior head support. In some embodiments, the posterior had support is attached to a superior end of the vest posterior portion. In some embodiments, the posterior head support is configured to provide support to a head of a patient (wearer) in a dorsal direction. In general, the posterior head support can be used on conjunction with the neck support or can be used independently of the neck support.

In some embodiments, the posterior head support comprises a bathing support member. In general, the bathing support member can be configured to permit a patient (wearer) to be placed in a reclining position in a bathing apparatus while contacting the bathing apparatus to prevent the patient (wearer) from being submerged in the bathing apparatus. In some embodiments, the bathing support member includes or is in the form of a hook or other curved or angled projection. The hook or other curved or angled projection can be configured to be placed over an edge of a tub or other similar bathing apparatus such that the posterior head support cannot enter the tub or bathing apparatus. The bathing support member can prevent a wearer's head from either sliding or tipping to a position that is below a surface of the water in the tub or bathing apparatus. This may be useful for preventing drowning or other injury related to a wearer's head or face from being under a surface of the water or being held at an angle such that water enters and/or does not leave a wearer's nose and/or mouth. For example, the posterior head support and bathing support member can prevent a wearer's head from tilting far enough back that water can enter the wearer's nose and thereby causing discomfort or injury.

In some embodiments, the posterior head support comprises a rigid posterior head peripheral support member disposed about a periphery of a flexible posterior head support platform.

In some embodiments, the rigid posterior head peripheral support member is formed of a plastic. In general, the plastic can be any suitable plastic as described above. In some embodiments, the plastic is a rigid plastic. In some embodiments, the flexible posterior head support platform is formed of a textile. In general, the textile can be a textile as described above. In some embodiments, the posterior head support is waterproof, water resistant, water repellant, quick-drying, or any combination of these. As the positioning apparatus may be used in water, for example, while bathing, swimming, in wet environments, in environments where a wearer may be splashed, or the like, or may be used in environments where the wearer may sweat. Having the posterior head support be waterproof, water resistant, water repellant, and/or quick-drying may be advantageous for wearer comfort, for ease of use, for longevity of the posterior head support, for cleaning, or for some other purpose.

In some embodiments, the posterior head support comprises a floatation aid. In general, the floatation aid can be any suitable structure or feature that causes the posterior head support to float in water. That is, the floatation aid is a buoyant structure or feature. For example, the floatation aid can be a structure formed of or filled with air, foam, or other buoyant material. The floatation aid can be attached to or disposed upon the posterior head support. In some embodiments, the floatation aid is reversibly attached to or disposed upon the posterior head support. That is, the floatation aid can be removed from the posterior head support. In some embodiments, the floatation aid can be attached to or disposed upon the rigid posterior head peripheral support member. For example, a tube of foam or a tube-shaped air-filled bladder can be attached to the rigid posterior head peripheral support member. In general, any number of floatation aids can be used.

In some embodiments, the pediatric positioning apparatus is devoid of electronic sensors. That is, there are no electronic components present in the support vest, neck support, or posterior head support.

The examples below are intended to further illustrate the positioning apparatus as well as protocols for preparing and/or using the positioning apparatus and are not intended to limit the scope of the claims.

### Examples

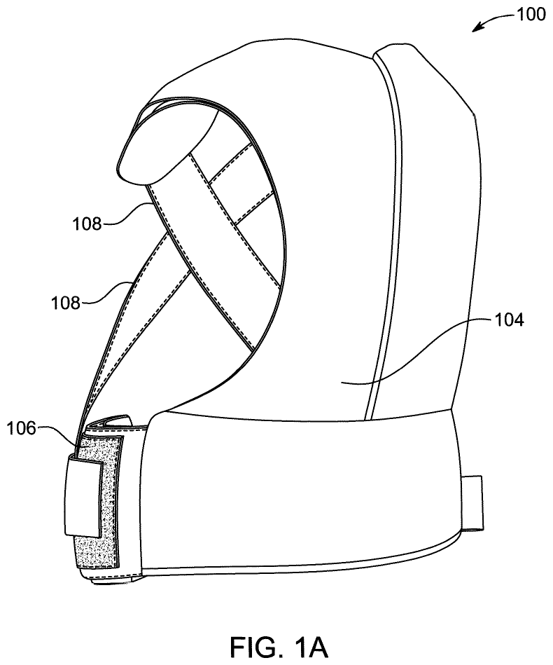

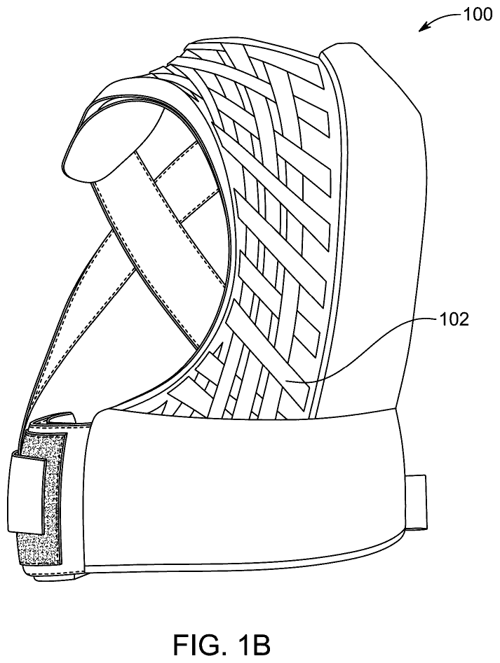

FIG. 1A shows a rear three-quarters view of a support vest according to an exemplary embodiment of the present disclosure. The support vest (100) is shown with the support vest cover intact (104) and covering the network of rigid support members. The support vest cover (104), one of the waist supports (106), and the anterior straps (108) are visible in the figure. FIG. 1B shows a rear three-quarters view of a support vest (100) with a portion of the vest posterior portion cover removed showing the network of rigid support members (102) according to an exemplary embodiment of the present disclosure. FIG. 1C shows a schematic depiction of a frontal view of a support vest (100) according to an exemplary embodiment of the present disclosure. The waist supports (106), the anterior straps (108), and the waist belt (110) are visible in the figure. FIG. 1D shows a schematic depiction of a side view of a support vest according to an exemplary embodiment of the present disclosure.

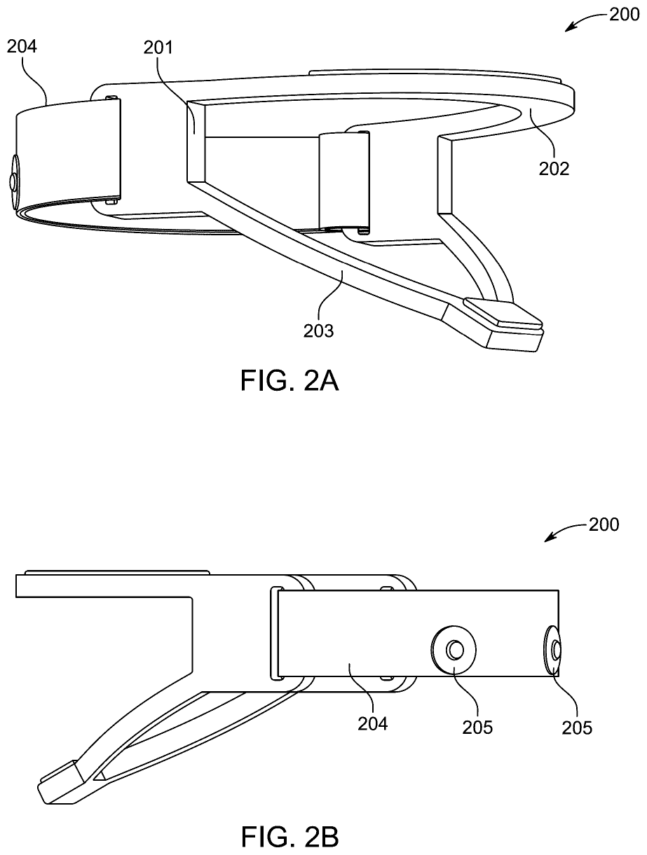

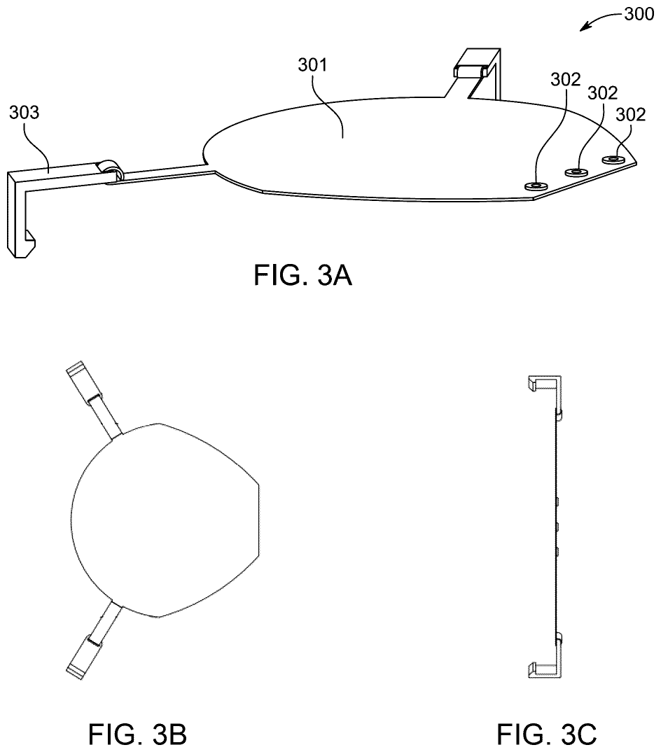

FIG. 2A shows a front three-quarters view of a neck support (200) according to an exemplary embodiment of the present disclosure. The anterior support jaw (201) formed of the superior jaw arc (202) and inferior jaw arc (203) is depicted along with the posterior neck strap (204). FIG. 2B shows a rear three-quarters view of a neck support (200) according to an exemplary embodiment of the present disclosure. Snaps (205) are depicted as being disposed on the posterior neck strap (204). These snaps can be configured to be used for attaching the posterior head support depicted in FIGS. 3A-3C to the neck support. FIG. 2C shows a schematic depiction of a top-down view of a neck support according to an exemplary embodiment of the present disclosure. FIG. 2D shows a schematic depiction of a side view of a neck support according to an exemplary embodiment of the present disclosure. FIG. 2E shows a schematic depiction of a frontal view of a neck support according to an exemplary embodiment of the present disclosure.

FIG. 3A shows a three-quarters view of a posterior head support (300) according to an exemplary embodiment of the present disclosure. The figure depicts a flexible posterior head support platform (301) with snaps (302) to be used for attaching the posterior head support depicted in FIGS. 3A-3C to the neck support depicted in FIGS. 2A-2D, and a pair of bathing support members (303). FIG. 3B shows a schematic depiction of a top-down view of a posterior head support according to an exemplary embodiment of the present disclosure. FIG. 3C shows a schematic depiction of a side view of a posterior head support according to an exemplary embodiment of the present disclosure.

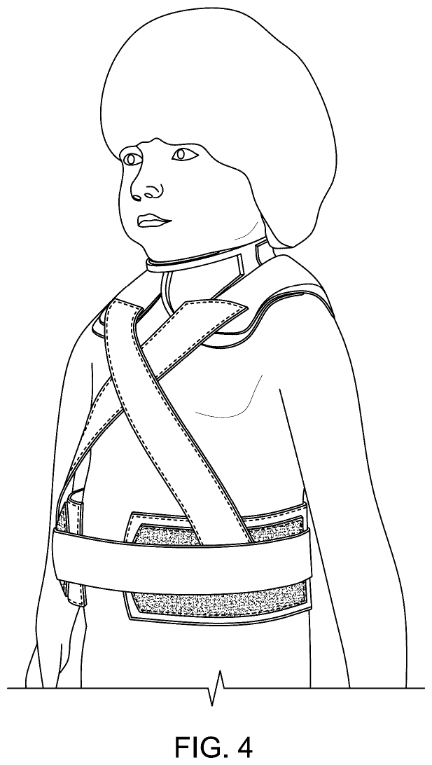

FIG. 4 shows a pediatric patient wearing a pediatric position apparatus according to an exemplary embodiment of the present disclosure.

Where a numerical limit or range is stated herein, the endpoints are included. Also, all values and subranges within a numerical limit or range are specifically included as if explicitly written out.

Numerous modifications and variations of the present invention are possible in light of the above teachings. It is therefore to be understood that, within the scope of the appended claims, the invention may be practiced otherwise than as specifically described herein.

1: A pediatric positioning apparatus, comprising
a support vest comprising
a vest posterior portion comprising a network of rigid support members and a vest posterior portion cover,
a pair of waist supports attached to the vest posterior portion and configured to wrap around at least 50% of a waist circumference of a patient, and
a pair of anterior straps attached to the vest posterior portion and configured to pass over shoulders and chest of a patient and secure to the waist supports; and
a neck support comprising
a posterior neck strap, and
an anterior support jaw comprising a superior jaw arc comprising a chin contact portion and an inferior jaw arc comprising a chest contact portion, the anterior support jaw being rigid and configured to contact a chin and a chest of a patient such that the anterior support jaw supports the chin of the patient in a ventral direction and prevents contact between the chin and the chest of the patient.

2. The pediatric positioning apparatus of claim 1, wherein the rigid support members are formed of a plastic.

3. The pediatric positioning apparatus of claim 1, wherein the rigid support members have a flattened contact surface, the contact surface being the surface oriented toward a patient wearing the support vest.

4. The pediatric positioning apparatus of claim 3, wherein the rigid support member has a support ridge disposed on a surface opposite the flattened contact surface.

5. The pediatric positioning apparatus of claim 1, wherein the network of rigid support members comprises a peripheral support member disposed about a periphery of the network of rigid support members.

6. The pediatric positioning apparatus of claim 1, wherein the vest posterior portion cover is formed of a textile.

7. The pediatric positioning apparatus of claim 6, wherein the textile is a synthetic textile formed of a polymer.

8. The pediatric positioning apparatus of claim 1, wherein the network of rigid support members is configured to be removable from the vest posterior portion cover.

9. The pediatric positioning apparatus of claim 1, wherein each waist support includes a rigid waist support member.

10. The pediatric positioning apparatus of claim 1, further comprising a waist belt attached to the pair of waist supports and configured such that the waist belt and pair of waist supports together wrap around an entirety of a waist circumference of a patient.

11. The pediatric positioning apparatus of claim 10, wherein the waist belt is attached to the pair of waist supports using hook-and-loop fasteners.

12. The pediatric positioning apparatus of claim 1, wherein the neck support is connected to the support vest by at least one selected from the group consisting of the anterior straps, a neck support connection strap attached to the support vest and the neck support, and a rigid neck support connection member attached to the support vest and the neck support.

13. The pediatric positioning apparatus of claim 1, wherein the anterior support jaw is formed of a plastic.

14. The pediatric positioning apparatus of claim 1, wherein the posterior neck strap is formed of a textile.

15. The pediatric positioning apparatus of claim 14, wherein the textile is a synthetic textile formed of a polymer.

16: The pediatric positioning apparatus of claim 1, further comprising a posterior head support attached to a superior end of the vest posterior portion, the posterior head support configured to provide support to a head of a patient in a dorsal direction.

17: The pediatric positioning apparatus of claim 16, wherein the posterior head support comprises a bathing support member configured to permit a patient to be placed in a reclining position in a bathing apparatus while contacting the bathing apparatus to prevent the patient from being submerged in the bathing apparatus.

18. The pediatric positioning apparatus of claim 16, wherein the posterior head support comprises a rigid posterior head peripheral support member disposed about a periphery of a flexible posterior head support platform.

19. The pediatric positioning apparatus of claim 16, wherein the rigid posterior head peripheral support member is formed of a plastic and the flexible posterior head support platform is formed of a textile.

20. The pediatric positioning apparatus of claim 1, wherein the pediatric positioning apparatus is devoid of electronic sensors.
